(19) United States
(12) Patent Application Publication
(10) Pub. No.: US 2025/0213889 A1
(43) Pub. Date: Jul. 3, 2025

(54) PARTICLE ACCELERATOR

(71) Applicant: Shanghai United Imaging Healthcare Co., Ltd., Shanghai (CN)

(72) Inventors: Zhaolong Zhang, Shanghai (CN); Peng Wang, Shanghai (CN); Gang Pan, Shanghai (CN); Zhiguo Ma, Shanghai (CN); Zexu Du, Shanghai (CN)

(21) Appl. No.: 19/004,586

(22) Filed: Dec. 30, 2024

(30) Foreign Application Priority Data
Dec. 29, 2023 (CN) 202311869082.9

(51) Int. Cl. A61N 5/10; H05H 7/22; H05H 9/04

(52) U.S. Cl. CPC A61N 5/1077; H05H 7/22; H05H 9/04; A61N 2005/1074; H05H 2007/225; H05H 2277/11

(57) ABSTRACT

A particle accelerator includes an acceleration cavity chain and at least one energy switch side cavity. The energy switch side cavity is communicated with two adjacent main cavities in the acceleration cavity chain through coupling openings respectively. The energy switch side cavity includes a pair of asymmetric side cavity noses arranged therein and an energy switch probe arranged at a top position thereof. The energy switch probe is configured to extend from the top position of the energy switch side cavity to a space between the pair of side cavity noses.

### CROSS-REFERENCE TO RELATED APPLICATIONS

The present disclosure claims priority to Chinese patent application No. 202311869082.9, titled “ACCELERATOR”, filed on Dec. 29, 2023, the entire content of which is incorporated herein by reference.

### TECHNICAL FIELD

The present disclosure relates to the technical field of particle acceleration, particularly to a particle accelerator.

### BACKGROUND

With the development of particle acceleration technology, various types of accelerators have emerged. For example, medical accelerators are used in biomedical applications for radiation therapy of tumors. Charged particle accelerators are electromagnetic devices that accelerate various types of charged particles to higher energies by artificially applying different electric fields. In order for charged particles to gain energy, an accelerating electric field is necessary. Depending on the type of particles being accelerated and the shape of the accelerating electric field, the trajectory of the particles during the acceleration process varies, which leads to the classification of various types of accelerators.

In conventional technology, accelerators that can output multiple energy levels of electron beams are mainly divided into three categories. The first type controls the angle or position of a phase-shifting plate in a side cavity to regulate the electric field strength in the downstream of an accelerator tube. The second type controls the insertion depth of a probe in the energy switch side cavity to change the amplitude of the electric field in the downstream cavities. The third type uses a phase switch to reverse the phase of the downstream electric field, turning it into a decelerating field. However, the phase-shifting plate requires a high motion control precision and has high manufacturing and processing requirements. Common probe-type energy switches are more commonly used for outputting therapeutic beams, but are less effective for lower-energy imaging beams. Changing the number of side cavities leads to fewer energy levels. Therefore, conventional accelerators cannot provide particle beams or imaging beams of varying energies.

### SUMMARY

The present disclosure provides a particle accelerator. The particle accelerator includes an acceleration cavity chain and at least one energy switch side cavity. The acceleration cavity chain includes a plurality of main cavities. The energy switch side cavity includes coupling openings and is communicated with two adjacent main cavities in the acceleration cavity chain through the coupling openings respectively. The energy switch side cavity includes a pair of asymmetric side cavity noses arranged therein and an energy switch probe arranged at a top position thereof. The energy switch probe is configured to extend from the top position of the energy switch side cavity to a space between the pair of side cavity noses.

In some embodiments, the energy switch side cavity has a semi-cylindrical structure. A rectangular plane of the semi-cylindrical structure is oriented toward and close to the acceleration cavity chain, and serves as a bottom surface of the energy switch side cavity. The coupling openings are formed on the bottom surface of the energy switch side cavity.

In some embodiments, the energy switch probe is configured to be able to extend to contact the bottom surface of the energy switch side cavity.

In some embodiments, the energy switch probe is configured such that an insertion depth of the energy switch probe into the energy switch side cavity is able to be changed to adjust a ratio of electric field strengths of the two adjacent main cavities.

In some embodiments, the particle accelerator further includes a measurement unit configured to measure an insertion depth of the energy switch probe in the energy switch side cavity.

In some embodiments, one of the main cavities has a power source interface, and in a particle acceleration direction, the energy switch side cavity is located downstream of the main cavity with the power source interface.

In some embodiments, the acceleration cavity chain includes a focusing section configured to accelerate and focus particles, and a size of the coupling opening of the energy switch side cavity closer to the focusing section is greater than a size of the coupling opening farther away from the focusing section.

In some embodiments, a distance between the pair of side cavity noses is greater than a diameter of the energy switch probe.

In some embodiments, the acceleration cavity chain includes a focusing section and a light-speed section. The focusing section is configured to accelerate and focus particles, such that the particles accelerated in the focusing section enter the light-speed section. The energy switch side cavity is located in the light-speed section.

In some embodiments, the acceleration cavity chain includes multiple periodic units, and an electromagnetic wave phase difference between two adjacent periodic units from the multiple periodic units is π.

In some embodiments, in a direction perpendicular to a particle acceleration direction, a centerline of the energy switch probe is offset or coincident with a centerline of the energy switch side cavity.

In some embodiments, the energy switch side cavity includes coupling tubes and connects and communicates with the main cavities through the coupling tubes, and the coupling tubes form the coupling openings, respectively.

In some embodiments, the pair of side cavity noses includes an upstream side cavity nose and a downstream side cavity nose in a particle acceleration direction. A distal end of the upstream side cavity nose is closer to a centerline of the energy switch side cavity than a distal end of the downstream side cavity nose.

In some embodiments, the distal end of the upstream side cavity nose is closer to a center dividing line between the two adjacent main cavities than the distal end of the downstream side cavity nose.

Details of one or more embodiments of the present disclosure are set forth in the following accompanying drawings and descriptions. Other features, objectives, and advantages of the present disclosure become obvious with reference to the specification, the accompanying drawings, and the claims.

### BRIEF DESCRIPTION OF THE DRAWINGS

FIG. 1 is a schematic diagram of a structure of a particle accelerator according to an embodiment of the present disclosure.

FIG. 2 is a schematic diagram showing a connection between an energy switch side cavity and two adjacent main cavities according to an embodiment of the present disclosure.

FIG. 3 is a schematic diagram showing changes of a field strength ratio by adjusting a position of an energy switch probe according to an embodiment of the present disclosure.

FIG. 4 is a schematic diagram showing a field strength distribution when an energy switch probe reaches a bottom of a switch side cavity according to an embodiment of the present disclosure.

FIG. 5 is a block diagram of a structure of a particle accelerator according to an embodiment of the present disclosure.

### DETAILED DESCRIPTION

In order to make the above objectives, features, and advantages of the present disclosure more apparent and easier to understand, the following detailed description of specific embodiments of the present disclosure will be given in conjunction with the accompanying drawings. Many specific details are described below to facilitate a thorough understanding of the present disclosure. However, the present disclosure can be implemented in many different ways other than those described here. Skilled persons in the field may make similar improvements without departing from the scope of the present disclosure. Therefore, the present disclosure is not limited to the specific embodiments disclosed below.

U.S. Patent No. 7,339,320B1 discloses an accelerator for accelerating a particle beam, which uses an energy switch with a probe to adjust the electric field strength within a cavity. The entire content disclosed in this patent is incorporated by reference into the present disclosure.

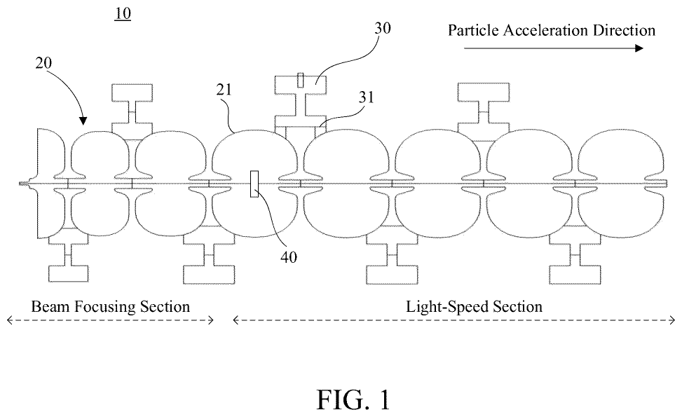

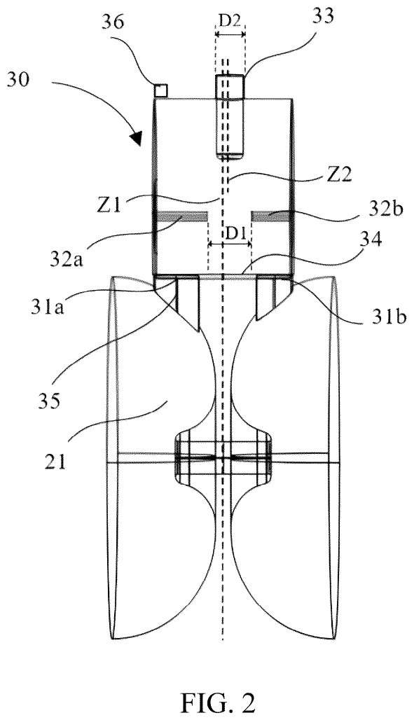

FIG. 1 shows a schematic structure of a particle accelerator according to an embodiment of the present disclosure. The particle accelerator 10 includes an acceleration cavity chain 20 and at least one energy switch side cavity 30. The acceleration cavity chain 20 includes a plurality of main cavities 21 that are sequentially connected. Referring to FIG. 2, each energy switch side cavity 30 is communicated with two adjacent main cavities 21 in the acceleration cavity chain 20 through coupling openings 31a, 31b. The energy switch side cavity 30 includes a pair of asymmetric side cavity noses 32a, 32b arranged therein and an energy switch probe 33 arranged on a top position thereof. The energy switch probe 33 can extend from the top position of the energy switch side cavity 30 into a space between the two side cavity noses 32a, 32b. The side cavity noses 32a, 32b and the energy switch probe 33 each function as a resonator for adjusting the resonant frequency of the energy switch side cavity 30. In the embodiment, by changing an insertion depth of the energy switch probe 33 in the energy switch side cavity 30, the distance between the energy switch probe 33 and the side cavity noses 32a, 32b is correspondingly altered, thereby changing the electric field strength in the energy switch side cavity 30. The magnetic field is then coupled into the coupling openings 31a, 31b, thereby altering the electric field energy in the downstream of the acceleration cavity chain 20.

Since electrons have a relatively low speed when they are first emitted from an electron gun, the space charge force causes significant particle diffusion. Therefore, the initial cavities in the acceleration cavity chain (commonly 2-4 cavities) are designed to be more suitable for accelerating low-speed particles, while also focusing the beam. These cavities are referred to as a focusing section of the acceleration cavity chain. The focusing section has a significant impact on the overall capture efficiency of the acceleration cavity chain (i.e., the ratio of an output current to an input current of the acceleration cavity chain). The focusing section adopts a segmented design, including a linear focusing section and a uniform focusing section. In the linear focusing section, a cavity phase velocity and the electric field increase linearly. In the uniform focusing section, the cavity phase velocity remains constant while the electric field decreases linearly. By optimizing the length of the uniform focusing section, the longitudinal acceleration phase of the electron beam can be easily controlled, thereby optimizing the capture efficiency. After passing through the focusing section, the electrons are accelerated to a speed close to the speed of light. Due to relativistic effects, the subsequent cavities cause a smaller change in the electron's speed (though the electron's kinetic energy is still increasing). The section used for accelerating particles with a speed close to the speed of light is referred to as the light-speed section.

Specifically, when the particle accelerator is started, with the asymmetrical side cavity noses, the energy switch side cavity first adjusts the electric field distribution within the acceleration cavity chain, achieving precise coupling between the acceleration cavity chain and the energy switch side cavity. This coupling ensures the smooth flow of particles during the acceleration process, minimizing energy loss and instability. The particle accelerator has several side cavities. These side cavities and the acceleration cavity chain jointly form an accelerator tube. One or more of the side cavities are used to control the electron beam energy, acting as energy switch side cavities. In practical applications, some of the energy switch side cavities have symmetrical noses, while side cavities at the focusing section have asymmetrical noses, but do not have energy switches. In the present disclosure, the side cavity noses of the energy switch side cavity where the energy switch probe is located are asymmetrical. Therefore, the energy switch side cavity with asymmetrical noses also constitutes an asymmetrical energy switch side cavity.

In the embodiments of the present disclosure, the energy switch side cavity 30 is connected to two adjacent main cavities 21 through coupling openings 31a, 31b, forming a seamless tube system that provides a stable passage for the particles. By adjusting the size of the coupling openings 31a, 31b on the acceleration cavity chain 20, the amplitudes of the acceleration electric fields in the main cavities 21 can be visually adjusted, such as the electric field amplitude at a central axis position. A pair of asymmetric side cavity noses 32a, 32b are arranged in the energy switch side cavity 30, which not only guides the particles along a specific path but also adjusts an electric field strength ratio of the main cavities 21 on both sides of the energy switch side cavity 30 due to the asymmetry. By altering the structures of the side cavity noses 32a, 32b during a design stage, the operator can achieve precise control over the kinetic energy of the particles, thereby meeting the needs of different experimental or application scenarios.

Referring to FIG. 2, the side cavity noses 32a, 32b are asymmetrically arranged relative to a centerline Z1 of the energy switch side cavity 30, which is perpendicular to a particle acceleration direction. In some embodiments, the side cavity noses 32a, 32b are located at the same height and extend laterally towards the centerline Z1 of the energy switch side cavity 30 from two opposite inner walls of the energy switch side cavity 30. However, the extension lengths of the noses 32a, 32b are not equal. For example, in the particle acceleration direction, the upstream side cavity nose 32a is longer, with its distal end closer to the centerline Z1 of the energy switch side cavity 30 than the downstream side cavity nose 32b, thereby adjusting the ratio of electric field amplitudes of the two main cavities 21 connected to the energy switch side cavity 30. It can also be understood as the ratio of the electric field strengths of the two main cavities being adjusted.

In the embodiments, since the centerline Z1 of the energy switch side cavity 30 coincides with a center dividing line between the two main cavities 21 connected to the energy switch side cavity 30, it indicates that the side cavity noses 32a, 32b are asymmetrically located relative to the center dividing line between the two adjacent main cavities 21. For example, the distal end of the upstream side cavity nose 32a is closer to the center dividing line between the two adjacent main cavities 21 than the distal end of the downstream side cavity nose 32b.

In the design stage, the length ratio between the upstream side cavity nose 32a and the downstream side cavity nose 32b can be adjusted as needed. The larger the length ratio between the upstream side cavity nose 32a and the downstream side cavity nose 32b, the smaller the electric field amplitude ratio between the corresponding main cavities 21.

In some embodiments, the side cavity noses 32a, 32b have metal column structures. It will be understood that, as needed, the side cavity noses 32a, 32b can have different set heights, design dimensions, or design structures. As a non-limiting example, in addition to the energy switch probe 33 shown in the figures, the energy switch may also include a driving device and a corrugated pipe (not shown), etc. The specific structure of the energy switch is well known to those skilled in the art and will not be described in detail here. By changing the insertion depth of the energy switch probe 33 into the energy switch side cavity 30, the energy switch can alter the electric field strength distributions at both sides of the acceleration cavity chain 20, for example, changing the electric field amplitude ratio of the adjacent main cavities 21 connected to the energy switch side cavity 30. In practical applications, the capture rate of the acceleration cavity chain 20 is also related to an injection voltage of the electron gun, which complements the adjustment of the energy switch. The above description only discusses the use of an energy switch in one side cavity. In practice, additional energy switch side cavities with energy switches can be provided at different positions to increase the adjustment capability. The energy switch probe 33 extends from the top of the energy switch side cavity 30 to the space between the two side cavity noses 32a, 32b, forming a precise control node. The energy switch directly affects the particle's motion in the path by releasing precisely controlled energy. When the depth at which the energy switch probe 33 enters the asymmetric energy switch side cavity 30 changes, it alters the electric field distribution in the acceleration cavity chain. Combined with changes in the input power, it allows the acceleration cavity chain 20 to output particle beams at different energy levels. Operators can dynamically adjust the particle speed in real time, enabling precise control over the acceleration process. This design provides the operators with great flexibility, allowing them to optimize and adjust parameters of particle acceleration in real time according to specific experimental requirements or research objectives.

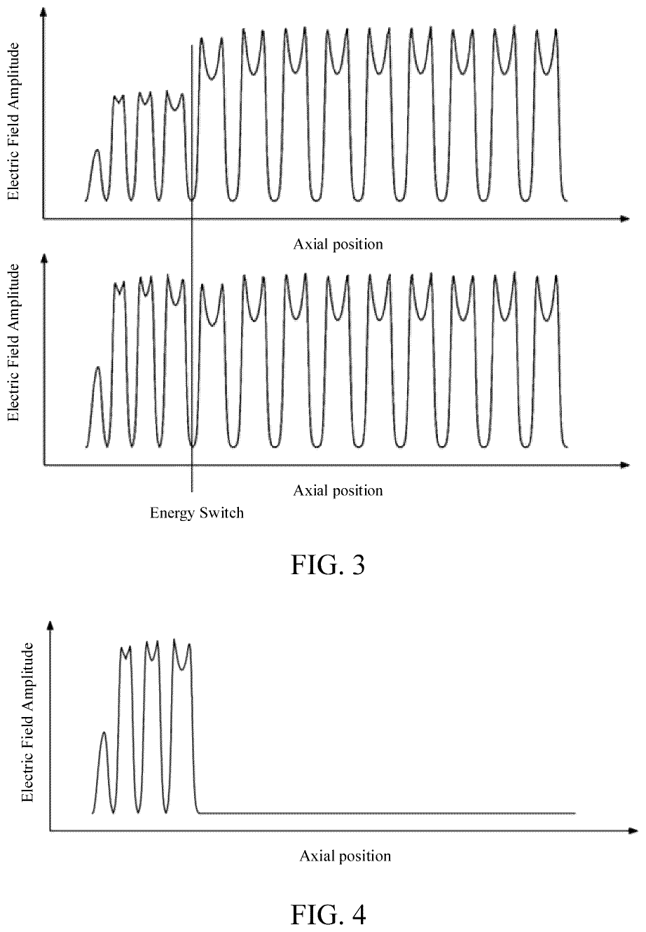

When a high-energy level is selected, i.e., when the energy switch probe 33 is not inserted, the electric field amplitude along a central axis of the acceleration cavity chain 20 is shown in the above subfigure in FIG. 3, and in this case, the electric field strength in each main cavity 21 depends on the design dimensions of the coupling openings 31a, 31b of each energy switch side cavity 30. In this case, the energy switch probe 33 has no effect. The electric field amplitude in the light-speed section is higher than in the focusing section, and thus this state is used for supplying high-energy beams. In this state, the power source generates and inputs a higher power into the acceleration cavity chain 20. It can be considered that the electric field in the focusing section at this time is the required electric field that can achieve a high capture rate. After the electrons pass through both the focusing and light-speed sections, they can be accelerated to the desired energy (high-energy level).

When a medium/low-energy level is selected and the energy switch probe is inserted to a certain depth, the overall electric field amplitude ratio is as shown in the below subfigure in FIG. 3. In this case, the electric field amplitude ratio between the focusing section and the light-speed section is greater. By reducing the input power, the overall actual electric field amplitude of the accelerator tube is lowered, while the actual electric field amplitude in the focusing section can remain the same as that in the high-energy level, thereby still achieving a high electron capture rate. However, at this time, the actual electric field amplitude in the light-speed section is lower than in the high-energy level. Ultimately, the desired energy for the electron beam is also obtained.

In an exemplary application, as shown in FIG. 3, combined with FIG. 1, the first four main cavities 21 are used as the focusing section. The electric field amplitude in this section is quite important for the capture rate of the entire accelerator tube. For example, up to thirty percent of the electrons can be accelerated and output. However, without the adjustment of the energy switch probe to regulate the electric field, the entire accelerator tube operates at a single energy level, with a fixed electric field distribution and a determined input energy, meaning that the output electron beam energy will also be fixed. After adding the energy switch probe 33, the electric field amplitude ratio between both sides can be varied by adjusting the insertion depth of the energy switch probe 33. The above subfigure in FIG. 3 depicts the electric field distribution along the particle acceleration direction when the energy switch probe 33 is not inserted. At this point, the input power A is adjusted to ensure that the electric field amplitude in the focusing section reaches the condition that maximizes the capture rate. This electric field amplitude in the focusing section is represented by P. In this case, a corresponding energy level is output.

The below subfigure in FIG. 3 depicts the electric field amplitude distribution along the particle acceleration direction when the energy switch probe 33 is inserted into the energy switch side cavity 30 by a certain distance. In this state, the ratio of the electric field amplitude between the focusing section and the light-speed section increases. If the input power is A, the electric field amplitude in the focusing section will not be in the condition that maximizes the capture rate, but may become P+X. Therefore, if the input power is reduced, for example, to A0, such that the electric field amplitude in the focusing section returns to the value when the probe was not inserted (as shown in the above subfigure in FIG. 3), the maximal capture rate can be achieved again. At this point, the capture rate of the accelerator tube is restored, while the electric field amplitude in the light-speed section and even the entire accelerator tube is actually reduced, meaning that the energy of the accelerated electrons will be lower than in the previous case. This will result in an output of a different energy level. Therefore, by synchronously adjusting the insertion depth of the energy switch probe and the input power, the accelerator tube can output electron beams with different energy levels while maintaining a high capture rate.

In some embodiments, the capture rate of the accelerator tube is also related to the injection voltage of the electron gun. In practical use, this variable can serve as a supplement to the energy switch adjustment capability. Similarly, in practice, new energy switch side cavities with energy switch probes can be added at different positions to increase the adjustment capability.

The high, medium, and low energy levels are all treatment beams, meaning they are energy levels used to generate X-rays for therapy through targeting or directly used for electron beam therapy. In the field of radiation therapy, to achieve better treatment effects, it is required that the accelerator tube can also output even lower energy imaging beams (below 1 MV or even at the kV level). Therefore, in some embodiments, a mode where the energy switch probe 33 is fully inserted is introduced, which corresponds to an imaging energy level. In this mode, the energy switch probe 33 reaches the bottom of the energy switch side cavity 30, short-circuiting the energy switch side cavity 30, causing the field strength in the subsequent main cavities 21 to become zero, thereby outputting the imaging beam, as shown in FIG. 4.

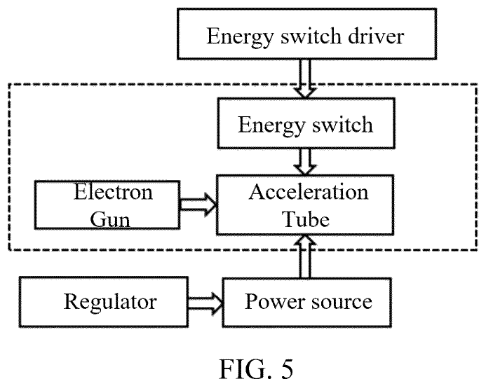

As shown in FIG. 5, which is a block diagram of the structure of the particle accelerator 10, the particle accelerator 10 includes an accelerator tube, an energy switch driving device, a power source, and a modulator. The energy switch driving device, e.g., a motor, adjusts the insertion depth of the energy switch probe 33 into the energy switch side cavity 30 based on instructions, thereby changing the energy of the electron beam output by the particle accelerator 10. The modulator controls the magnitude of the input power provided by the power source to the particle accelerator 10.

According to the embodiments of the present disclosure, the asymmetric energy switch side cavity, together with the energy switch probe, makes the electric field distribution inside the acceleration cavity chain adjustable. The asymmetric energy switch side cavity works with the power source, allowing the acceleration cavity chain to output particle beams with different energy levels. The energy switch is located at the top of the energy switch side cavity, and the energy switch probe of the energy switch can precisely extend between the two side cavity noses, enabling the operator to finely adjust the acceleration process according to requirements. It not only improves the acceleration efficiency but also makes the treatment process more controllable and allows for precise adjustments, providing particle beams with different energies, including therapeutic beams and imaging beams.

In some embodiments, the energy switch side cavity 30 has a semi-cylindrical structure. The rectangular plane of the semi-cylindrical structure serves as the bottom surface 34 of the energy switch side cavity 30, and is oriented toward and close to the acceleration cavity chain 21. It should be noted that the semi-cylindrical structure defined here may include a perfect semi-cylindrical shape or an approximate semi-cylindrical shape. The coupling openings 31a, 31b are formed on the bottom surface 34 of the energy switch side cavity 30.

It can be understood that, as needed, the structure of the energy switch side cavity 30 can be adjusted. For example, it could be cylindrical, elliptical cylindrical, or other shapes.

Referring to the embodiment shown in FIGS. 1-2, the energy switch side cavity 30 includes coupling tubes 35, through which the energy switch side cavity 30 connects and communicates with the main cavity 21. The coupling tubes 35 form the coupling openings 31a, 31b, respectively.

It should be understood that in other embodiments, the coupling tube may not be used. Instead, the energy switch side cavity may be directly assembled with the main cavity, with coupling openings formed at the connection between the two.

In the embodiments, the energy switch side cavity 30 is designed with a semi-cylindrical structure. The rectangular plane of the semi-cylindrical structure serves as the bottom surface 34 of the energy switch side cavity 30, and is oriented toward and close to the main cavities 21. The coupling openings 31a, 31b are formed on the bottom surface 34 of the energy switch side cavity 30, enabling effective connection between the energy switch side cavity 30 and the main cavities 21. This ensures tight coupling between the energy switch side cavity 30 and the main cavities 21, thereby ensuring that the particle accelerator 10 has a better particle capture rate and improves the energy control of the electron beam in the particle accelerator 10. This is further beneficial for enhancing the precision of particle therapy in medical applications. In some embodiments, the energy switch probe 33 can extend to contact the bottom surface 34 of the energy switch side cavity 30.

Specifically, the energy switch probe 33 is configured to extend to contact the bottom surface 34 of the energy switch side cavity 30. The energy switch probe 33 directly contacts the bottom surface 34 of the energy switch side cavity 30, achieving highly precise control of the energy switch. When the energy switch probe 33 reaches the bottom of the energy switch side cavity 30, it short-circuits the energy switch side cavity 30, causing the frequency of the energy switch side cavity 30 to become detuned. This results in the electric field in the subsequent main cavity 21 being zero, preventing particle acceleration in the main cavity 21, which further lowers the particle energy and outputs, for example, a lower energy imaging beam. In practical operation, when the energy switch is activated, the energy switch probe 33 extends into the energy switch side cavity 30 and makes contact with the bottom surface 34, so that the energy switch probe 33 directly contacts the energy switch side cavity 30. By the energy switch probe 33 closely contacting the bottom surface 34 of the energy switch side cavity 30, the energy switch enables highly precise control over the particle energy inside the particle accelerator 10, allowing for real-time response and adjustment of the particle beam.

Through the direct contact between the energy switch probe 33 and the bottom surface 34 of the energy switch side cavity 30, highly precise control over the particle beam energy inside the particle accelerator 10 is achieved. The fine control allows for adaptation to different treatment needs and variations, enabling doctors to flexibly adjust the energy of the particle beam according to specific situations. This capability helps to better tailor the treatment to different types of tumors or lesions, enhancing the personability level.

In some embodiments, the insertion depth of the energy switch probe 33 into the energy switch side cavity 30 can be controlled to adjust the ratio of the electric field strengths between the two adjacent main cavities 21 connected to the energy switch probe 33.

The primary function of the energy switch driving device is to control the insertion depth of the energy switch probe 33 into the energy switch side cavity 30, in order to adjust the electric field strength ratio between two adjacent main cavities 21. In actual operation, when the driving device is activated, it precisely controls the insertion depth of the energy switch probe 33, achieving the adjustment of the electric field strength inside the energy switch side cavity 30. This control process is adjusted based on specific treatment needs and the properties of the particle beam, ensuring that a precise electric field strength ratio is achieved between the two adjacent main cavities 21.

In the embodiments, by changing the insertion depth of the energy switch probe 33 into the energy switch side cavity 30 to adjust the electric field strength ratio between the two adjacent main cavities 21, the doctor can flexibly adjust the energy of the particle beam according to the patient's specific condition and treatment plan, thereby optimizing the treatment effect to the greatest extent. This fine control of the electric field helps improve the performance of the particle accelerator 10, making radiation therapy more precise and personalized, and ultimately providing a more effective treatment plan for the patient.

In some embodiments, the energy switch includes a measurement unit 36. The measurement unit 36 is configured to measure the insertion depth of the energy switch probe 33 into the energy switch side cavity 30.

Specifically, the measurement unit 36 is configured to measure the insertion depth of the energy switch probe 33 into the energy switch side cavity 30, thereby enabling precise monitoring and control of the position of the energy switch probe 33. During operation, the measurement unit 36 may include an optical sensor or other types of displacement sensors, which record and provide real-time feedback on the position of the energy switch probe 33. This measurement allows for highly accurate determination of the insertion depth of energy switch probe 33 into the energy switch side cavity 30, providing real-time data feedback to the doctor. The real-time monitoring information can be used to adjust the driving device, ensuring precise control of the energy switch probe 33, which in turn adjusts the electric field strength in the energy switch side cavity 30.

In the embodiments, through the application of the measurement unit 36, the particle accelerator 10 can more precisely sense and adjust the position of the energy switch probe 33 during operation, thereby achieving precise control of the particle beam. This helps improve the stability and performance of the particle accelerator 10, providing high levels of precision and safety for particle therapy in the medical field.

In some embodiments, the particle accelerator 10 also includes a power source. The power source provides the energy required for particle acceleration. The power source is connected to the acceleration cavity chain 20 through a waveguide. One of the main cavities 21 in the acceleration cavity chain 20 includes a power source interface 40, through which the power source connects to the acceleration cavity chain 20. The output power is adjustable. The power source includes a magnetron or a klystron, which can control the frequency and power of the output electromagnetic waves within a certain range through a modulator, thereby regulating the particle acceleration process. During actual operation, the power source directly influences the particle acceleration process in the acceleration cavity chain 20 by adjusting the microwave power. By increasing or decreasing the microwave power, doctors can precisely adjust the energy level of the particle beam. This adjustment process is highly flexible and can be fine-tuned according to the patient's specific condition and treatment plan.

In the embodiments, the presence of the power source enables the particle accelerator 10 to accommodate different types of tumors or lesions, offering more flexible and precise treatment options. The modulation of microwave power allows for an immediate response to the particle beam, ensuring stability and precision during the treatment process. Overall, the application of the power source in this hardware configuration enhances the functionality of the particle accelerator 10, providing a higher level of customization and adjustment capability for particle therapy in the medical field.

In some embodiments, the energy switch side cavity 30 is located in the light-speed section, and the size of the coupling opening 31a closer to the focusing section is larger than the size of the coupling opening 31b farther away from the focusing section.

Specifically, the size of the coupling opening 31a closer to the focusing section is designed to be larger than the size of the coupling opening 31b farther away from the focusing section. In actual operation, this design takes into account the focusing effect of particles in the focusing section, so the coupling opening 31a closer to the focusing section is relatively larger, which helps achieve a smoother transition when the particle beam passes through the main cavity 21, thereby improving the particle density and focusing effects. In contrast, the coupling opening 31b farther away from the focusing section is smaller, which helps maintain a lower particle density to accommodate the increasing speed of particles during the acceleration process. This differential size design optimizes the entire particle accelerator 10, ensuring smooth transitions between different sections and ultimately improving the performance and efficiency of the accelerator 10.

In the embodiments, the differential design of the sizes of coupling openings 31a, 31b takes into account the focusing effect, allowing the particle accelerator 10 to operate more smoothly, providing patients with precise and safe treatments.

In some embodiments, the distance D1 between the two side cavity noses 32a, 32b is greater than the diameter D2 of the energy switch probe 33.

In some embodiments, the distance between the two side cavity noses 32a, 32b is set to be greater than the diameter of the energy switch probe 33. In practice, this design considers the diameter of the energy switch probe 33 and the relative positions of the side cavity noses 32a, 32b. The distance between the two side cavity noses 32a, 32b is greater than the diameter of the energy switch probe 33, ensuring that the energy switch probe 33 can move freely between the side cavity noses 32a, 32b without obstruction. This design plays a crucial role in particle acceleration and guiding, ensuring that the energy switch probe 33 can move freely between the side cavity noses 32a, 32b, allowing flexible control of the particle beam. At the same time, the two side cavity noses 32a, 32b may be designed to have different lengths, further enhancing the diversity of the design and increasing the degree of freedom in the design process. This difference has an important impact on the guidance and focusing of the particle beam, especially under different energy and treatment requirements. By adjusting the sizes of the side cavity noses 32a, 32b, the flow characteristics of the particle beam can be more precisely customized and optimized, thereby improving the adaptability and treatment effectiveness of the particle accelerator 10. In the embodiments, by configuring the distance between the side cavity noses 32a, 32b and the sizes of the side cavity noses 32a, 32b, the free movement of the energy switch probe 33 is ensured, while providing the particle accelerator 10 with a more flexible and diversified operation method, contributing to the personability level.

In the embodiment shown in FIG. 2, a centerline Z2 of the energy switch probe 33, which is perpendicular to the particle acceleration direction, is offset from the centerline Z1 of the energy switch side cavity 30. This offset helps to better match the gap between the two side cavity noses 32a, 32b. It is understood that, in other embodiments, the centerline of the energy switch probe 33, which is perpendicular to the particle acceleration direction, can coincide with the centerline of the energy switch side cavity 30, while ensuring that the two side cavity noses 32a, 32b do not interfere with the movement of the energy switch probe 33.

In a non-limiting embodiment, the energy switch side cavity 30 is located in the light-speed section of the acceleration cavity chain 20.

Specifically, the energy switch side cavity 30 is located in the light-speed section of the acceleration cavity chain 20. This design takes into account the special characteristics of the light-speed section during particle acceleration, aiming to optimize the guidance and control of the particles. Specifically, the speed of the particles in the light-speed section approaches the speed of light, and arranging the energy switch side cavity 30 in this section helps make full use of the particle's characteristics in high-speed motion, allowing the energy switch side cavity 30 to precisely and flexibly control the particles. The presence of the energy switch side cavity 30 allows for more precise guidance of particles in the light-speed section, adjusting their trajectory and speed to accommodate different types of treatment needs.

In a non-limiting embodiment, an operating mode of the acceleration cavity chain 20 is set to the π/2 mode.

Specifically, the operating mode of the acceleration cavity chain 20 is set to the π/2 mode. The choice of operating mode relates to the phase changes of the particles during the acceleration process. Since the speed of electromagnetic waves in a vacuum is the speed of light, to synchronize the phase velocity of the electromagnetic field with the charged particles, the acceleration cavity chain is designed with different types of periodic structures. For the same structure, depending on the phase shift of the electromagnetic field during one periodic unit, different modes can be defined. The π/2 mode indicates that the phase difference of the electromagnetic wave between adjacent periodic units is π/2. In this case, one electromagnetic wave period requires four periodic units. When the acceleration cavity chain operates in the π/2 mode, half of the cavities are at positions where the electric field magnitude is zero, which does not affect the charged particles, known as coupling cavities. Therefore, a dual-period structure is introduced that compresses the coupling cavities and moves them off the axis to increase the acceleration efficiency per unit length of the entire accelerator tube, called the side-coupled accelerating structure. In this structure, the coupling cavities are referred to as energy switch side cavities, and the accelerating cavities on the axis are referred to as main cavities. In the present disclosure, adjacent halves of the main cavity and the side cavity between them form a periodic unit. The operating phase of the acceleration cavity chain 20 is set to π, i.e., an electromagnetic wave phase difference between two adjacent periodic units from the multiple periodic units is π, which means that after the particle undergoes acceleration through one periodic unit, the phase shifts by 180 degrees. The operating mechanism of the π/2 mode may involve introducing specific phase control devices as the particles pass through the acceleration cavity chain 20, causing the phase of the particles to change in the π/2 mode. The choice of this operating mode is related to the acceleration, focusing, and guiding of particles. The π/2 mode may have a special effect on the particles' trajectories and speeds during the acceleration process, thereby optimizing the performance of the particle accelerator 10. The application of this mode can better adapt to specific treatment needs and improve the effectiveness and precision of particle therapy.

In the embodiments, the π/2 mode provides a specific operating mode for the particle accelerator 10, which regulates the particle's motion state through phase changes. This is expected to enable more flexible and optimized particle control during treatment.

In some embodiments, each energy switch side cavity 30 is located downstream of the main cavity 21 with the power source interface 40.

Specifically, each energy switch side cavity 30 is located downstream of the main cavity 21 connected to the power source. That is, in the particle acceleration direction, the main cavity 21 connected to the energy switch side cavity 30 is located downstream of the main cavity 21 connected to the power source, or in other words, the energy switch side cavity 30 is located downstream of the coupling cavity corresponding to the main cavity 21 connected to the power source. In practical operation, this configuration arranges the energy switch side cavity 30 after the position where the main cavity 21 is connected to the power source, which ensures that the energy switch side cavity 30 can accurately guide and control the particle beam after the microwave power is adjusted. When the microwave power changes under the control of the power source, the position of the energy switch side cavity 30 can better adapt to these changes, ensuring the stable acceleration and guiding process of the particles.

In the embodiments, the design of locating the energy switch side cavity 30 downstream of the main cavity 21 connected to the power source helps improve the overall stability and performance of the particle accelerator 10. This configuration not only optimizes the hardware structure but also enhances the accuracy and adaptability of the particle accelerator 10 during the treatment process, providing patients with a more reliable treatment plan.

It should be understood that the technical features of the above embodiments can be combined in any way. For the sake of simplicity, not all possible combinations of the technical features in the above embodiments are described. However, as long as the combination of these technical features does not result in contradictions, it should be considered within the scope of this description.

The embodiments described above only represent a few implementations of the present disclosure, which are relatively specific and detailed, but should not be understood as limiting the scope of the invention's patent rights. It should be noted that, for a person skilled in the art, without departing from the spirit of the present disclosure, various modifications and improvements can be made, all of which fall within the protection scope of the present disclosure. Therefore, the scope of protection for this patent application should be determined by the appended claims.

In the description of the present disclosure, it should be understood that terms such as “center”, “longitudinal”, “lateral”, “length”, “width”, “thickness”, “up”, “down”, “front”, “rear”, “left”, “right”, “vertical”, “horizontal”, “top”, “bottom”, “inner”, “outer”, “clockwise”, “counterclockwise”, “axial”, “radial”, “circumferential”, and other directional or positional terms are based on the orientation or positional relationships shown in the drawings, and are used solely for convenience in describing and simplifying the description of the present disclosure. These terms should not be interpreted as indicating or implying that the device or component in question must have a specific orientation or be constructed and operated in a specific orientation. Therefore, these terms should not be understood as limitations on the scope of the present disclosure.

Additionally, the terms “first” and “second” are used only for descriptive purposes and should not be understood as indicating relative importance or implicitly suggesting the quantity of the features indicated. Therefore, features defined as “first” and “second” may explicitly or implicitly include at least one of the respective features. In the present disclosure, the term “plurality” means at least two, such as two, three, etc., unless specifically stated otherwise.

In the present disclosure, unless explicitly stated otherwise, terms such as “install”, “connected”, “coupled”, “fixed”, etc., should be broadly understood. For example, these can refer to a fixed connection, a detachable connection, or integration into one piece; they can refer to mechanical connections, electrical connections, or even fluid connections; they can refer to direct connections or indirect connections through an intermediary; they can also refer to communication between two elements or the interaction between them, unless otherwise explicitly defined. For those skilled in the art, the specific meaning of these terms in the present disclosure can be understood based on the context.

In the present disclosure, unless explicitly stated otherwise, the term “on” or “above” in relation to a first feature and a second feature can mean that the first and second features are in direct contact, or they are in indirect contact through an intermediary. Additionally, “above”, “on top of”, or “above” the second feature can indicate that the first feature is either directly above the second feature, at an inclined angle above it, or simply that the first feature has a higher vertical position compared to the second feature. Similarly, “below”, “under”, or “beneath” the second feature can mean that the first feature is directly below, or at an inclined angle below the second feature, or simply that the first feature has a lower vertical position compared to the second feature.

It should be noted that when an element is referred to as “fixed to” or “arranged on” another element, it can either be directly on the other element or can be located with an intermediary element in between. When one element is said to be “connected” to another element, it can be directly connected or may also involve an intermediary element. The terms “vertical”, “horizontal”, “above”, “below”, “left”, “right”, and similar expressions used in the present disclosure are intended for illustration purposes only and do not indicate a single, limiting method of implementation

The technical features of the embodiments described above can be combined in any way. For the sake of simplicity, not all possible combinations of the technical features in the above embodiments are described. However, as long as these combinations do not conflict with each other, they should be considered within the scope disclosed in this specification.

The embodiments described above only represent a few implementations of the present disclosure, and the descriptions are relatively specific and detailed, but they should not be construed as limiting the scope of the patent application. It should be noted that for those skilled in the art, without departing from the concept of the present disclosure, several modifications and improvements can still be made, and these should also fall within the protection scope of the present disclosure. Therefore, the protection scope of the present disclosure should be determined by the appended claims.

What is claimed is:

1. A particle accelerator, comprising an acceleration cavity chain and at least one energy switch side cavity, the acceleration cavity chain comprising a plurality of main cavities, wherein the energy switch side cavity comprises coupling openings and is communicated with two adjacent main cavities in the acceleration cavity chain through the coupling openings respectively, the energy switch side cavity comprises a pair of asymmetric side cavity noses arranged therein and an energy switch probe arranged at a top position thereof, and the energy switch probe is configured to extend from the top position of the energy switch side cavity to a space between the pair of side cavity noses.

2. The particle accelerator according to claim 1, wherein the energy switch side cavity has a semi-cylindrical structure, and a rectangular plane of the semi-cylindrical structure is oriented toward and close to the acceleration cavity chain, and serves as a bottom surface of the energy switch side cavity, the coupling openings being formed on the bottom surface of the energy switch side cavity.

3. The particle accelerator according to claim 2, wherein the energy switch probe is configured to be able to extend to contact the bottom surface of the energy switch side cavity.

4. The particle accelerator according to claim 3, wherein the energy switch probe is configured such that an insertion depth of the energy switch probe into the energy switch side cavity is able to be changed to adjust a ratio of electric field strengths of the two adjacent main cavities.

5. The particle accelerator according to claim 1, further comprising a measurement unit configured to measure an insertion depth of the energy switch probe in the energy switch side cavity.

6. The particle accelerator according to claim 1, wherein one of the main cavities has a power source interface, and in a particle acceleration direction, the energy switch side cavity is located downstream of the main cavity with the power source interface.

7. The particle accelerator according to claim 1, wherein the acceleration cavity chain comprises a focusing section configured to accelerate and focus particles, and a size of the coupling opening of the energy switch side cavity closer to the focusing section is greater than a size of the coupling opening farther away from the focusing section.

8. The particle accelerator according to claim 1, wherein a distance between the pair of side cavity noses is greater than a diameter of the energy switch probe.

9. The particle accelerator according to claim 1, wherein the acceleration cavity chain comprises a focusing section and a light-speed section, the focusing section being configured to accelerate and focus particles, such that the particles accelerated in the focusing section enter the light-speed section, the energy switch side cavity being located in the light-speed section.

10. The particle accelerator according to claim 1, wherein the acceleration cavity chain comprises multiple periodic units, and an electromagnetic wave phase difference between two adjacent periodic units from the multiple periodic units is π.

11. The particle accelerator according to claim 1, wherein in a direction perpendicular to a particle acceleration direction, a centerline of the energy switch probe is offset or coincident with a centerline of the energy switch side cavity.

12. The particle accelerator according to claim 1, wherein the energy switch side cavity comprises coupling tubes and connects and communicates with the main cavities through the coupling tubes, and the coupling tubes form the coupling openings, respectively.

13. The particle accelerator according to claim 1, wherein the pair of side cavity noses comprises an upstream side cavity nose and a downstream side cavity nose in a particle acceleration direction, a distal end of the upstream side cavity nose being closer to a centerline of the energy switch side cavity than a distal end of the downstream side cavity nose.

14. The particle accelerator according to claim 13, wherein the distal end of the upstream side cavity nose is closer to a center dividing line between the two adjacent main cavities than the distal end of the downstream side cavity nose.
